(19) United States
(12) Patent Application Publication
(10) Pub. No.: US 2006/0251610 A1
(43) Pub. Date: Nov. 9, 2006

(54) Structure for separation of physiologically active agent and method for recovering physiologically active agent

(75) Inventor: Kazumichi Nakahama, Tokyo (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/412,866

(22) Filed: Apr. 28, 2006

(30) Foreign Application Priority Data
May 6, 2005 (JP) 2005-134985

(51) Int. Cl. C12Q 1/68; A61K 31/785

(52) U.S. Cl. 424/78.3; 435/6

(57) ABSTRACT

The present invention is directed to separate a physiologically active agent accurately. Then, the present invention provides a structure for separation of a physiologically active agent, comprising a substrate, a substance exhibiting affinity for the physiologically active agent, and a block polymer composed of a segment having a lower critical solution temperature (LCST) and a hydrophilic segment, in which the substance exhibiting affinity and the block polymer are bound to the substrate.

### BACKGROUND OF THE INVENTION

1. Field of the Invention

The present invention relates to a structure for separation of a physiologically active agent and a method for recovering a physiologically active agent. In particular, application as a separating agent for chromatography is expected.

2. Related Background Art

Recently, with a rapid progress of genetic engineering and the like, application of physiologically active agents (such as physiologically active peptides, proteins and DNA) to various fields including pharmaceutical products has been expected. Under such circumstance, separation methods and purification methods for physiologically active agents have been extremely important.

In particular, there is a growing need for a technique of separating and purifying a physiologically active agent without damaging its activity.

As a technique for separating and purifying a physiologically active agent, liquid chromatography is known. Among others, affinity chromatography has been widely used, because it can selectively recover a target physiologically active agent. In the affinity chromatography, separation can be made using specific interaction between biological substances. More specifically, separation is performed using, as a packing material, a structure to which a substance having affinity (hereinafter referred to as an affinity ligand) for a physiologically active agent is bound. However, in a conventional affinity chromatography, an acidic or alkaline eluant and a large amount of a competitive agent must be added to recover a separated physiologically active agent, causing various problems, including complicated operation and lowered activity of a physiologically active agent.

To solve these problems, Japanese Patent Application Laid-Open No. H07-318851 proposes a method for recovering a physiologically active agent by affinity chromatography conducted under mild solvent conditions that do not decrease the activity thereof. The invention is characterized in that silica particles to which a polymer having a lower critical solution temperature (hereinafter referred to as “LCST”) and an affinity ligand are independently bound are used as a column packing material. The column packing material binds to a physiologically active agent at a temperature equal to or higher than the LCST of the polymer, since the polymer shrinks at the temperature so that the affinity ligand is exposed on the surface of the packing material. On the other hand, at a temperature equal to or less than the LCST, the polymer melts and covers the affinity ligand. Therefore, the physiologically active agent cannot bind to the column packing material. Because of these characteristics, the retention of a physiologically active agent can be controlled without changing a solvent composition of a packing solution when the affinity chromatography packed with such a column packing material.

However, in the method disclosed in Japanese Patent Application Laid-Open No. H07-318851, a polymer bound to a column packing material non-specifically adsorbs to another physiologically active agent 4a at a temperature equal to or higher than the LCST (FIG. 4B). In the figure, reference numeral 3a represents a physiologically active agent and 1b represents a polymer. This is because the polymer itself is hydrophobic at the temperature equal to or higher than the LCST and a hydrophobic interaction takes place between the polymer and a hydrophobic part of physiologically active agent 4a, leading to the non-specific adsorption. As a consequence, a separated physiologically active agent 3a and the physiologically active agent 4a are randomly adsorbed to the polymer 1b. When the temperature is reduced to a temperature lower than the LCST in this state, the separated target physiologically active agent 3a and another physiologically active agent 4a are to be recovered. In short, inactivation of the physiologically active agent can be reduced; however, it is difficult to separate the predetermined (target) physiologically active agent accurately. Under such circumstances, the present invention is directed to separating a predetermined physiologically active agent accurately.

### SUMMARY OF THE INVENTION

According to the present invention, there is provided a structure for separation of a physiologically active agent comprising

a substrate,

a substance exhibiting affinity for the physiologically active agent, and

a block polymer composed of a segment having a LCST and a hydrophilic segment,

in which the substance exhibiting affinity and the block polymer are bound to the substrate.

According to the present invention, there is also provided a method for recovering a physiologically active agent comprising the steps of:

preparing the structure for separation described above and a solution containing a physiologically active agent;

soaking the structure for separation in the solution set at a temperature equal to or lower than the LCST, thereby adsorbing the physiologically active agent to the structure for separation; and

recovering the physiologically active agent adsorbed to the structure.

### BRIEF DESCRIPTION OF THE DRAWINGS

FIG. 1 is an illustration showing a structure for separation of a physiologically active agent, in which an affinity ligand and a block polymer are bound to a substrate;

FIGS. 2A and 2B are illustrations showing the behavior of a structure for separation of a physiologically active agent in response to a change in temperature;

FIGS. 3A, 3B and 3C are illustrations showing how to recover a physiologically active agent using a structure for separation of a physiologically active agent; and

FIGS. 4A, 4B and 4C are illustrations showing a conventional structure for separation.

### DETAILED DESCRIPTION OF THE PREFERRED EMBODIMENT

The present invention will be more specifically described.

First, a structure for separation of a physiologically active agent according to the present invention will be described with the reference to the accompanying drawings.

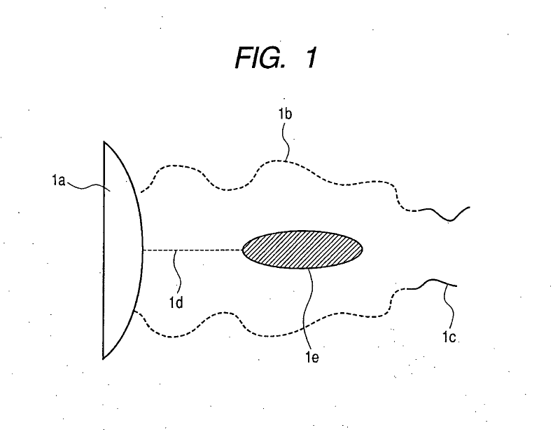

FIG. 1 is an illustration schematically showing a structure for separation of a physiologically active agent according to the present invention.

The structure for separation of a physiologically active agent according to the present invention is characterized in that a block polymer and an affinity ligand 1e are independently bound to the surface of a substrate 1a. The block polymer is composed of a segment 1b having an LCST and a hydrophilic segment 1c. Furthermore, the affinity ligand 1e usually binds to the surface of the substrate 1a via a spacer 1d. However, the affinity ligand 1e may be directly bound to the substrate 1a as long as the affinity ligand 1e does not lose its function.

Next, LCST will be explained. When temperature of a two-component solution separating into two discrete layers is increased or decreased, the compositions of the two phases gradually come closer. A temperature at which the compositions of the two phase are the same and a stable single phase is formed is referred to as “critical solution temperature.” A temperature at which two phases are changed into a single phase by lowering temperature is called as “lower critical solution temperature (LCST).” On the other hand, a temperature at which two phases are changed into a single phase by increasing temperature is called as “upper critical solution temperature (UCST).” The present invention purposely uses a block polymer having an LCST.

The polymer bound to the surface of the substrate 1a illustrated in FIG. 1 is a diblock polymer composed of two types of segment chains. However, as long as a polymer is composed of a segment 1b having an LCST and a hydrophilic segment 1c, a triblock polymer having three types of segments or a multi-block polymer having more than three type of segments may be used within the range where the same effect as in the diblock polymer is expected.

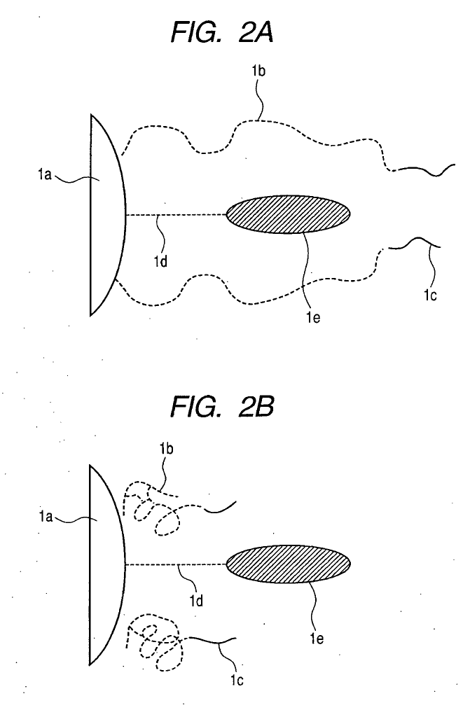

FIGS. 2A and 2B show changes in status of a structure for separation of a physiologically active agent according to the present invention at a temperature equal to or less than the LCST and equal to or higher than the LCST, respectively. FIG. 2A shows a structure for separation of a physiologically active agent according to the present invention at a temperature equal to or less than the LCST. In the temperature range, the segment 1b having an LCST is well dissolved in a solution and an affinity ligand 1e is covered with a block polymer formed of the segment 1b having an LCST and the segment 1c. On the other hand, FIG. 2B shows a structure for separation of a physiologically active agent according to the present invention at a temperature equal to or higher than the LCST. In the temperature range, the segment 1b having an LCST is hydrophobic and shrinks and the affinity ligand 1e is thus exposed on the surface of the material. Note that the hydrophilic segment 1c is well dissolved in absolution either in FIG. 2A and FIG. 2B and plays a role of maintaining the hydrophilic degree of the surface of the material.

Now, a structure of the present invention will be explained in more detail together with a method of manufacturing the same.

[Substrate]

As a substrate 1a according to the present invention, various materials such as organic materials and inorganic materials or hybrid materials of these may be used. The substrate 1a preferably has a functional group capable of chemically binding to a block polymer composed of a segment 1b having an LCST and a hydrophilic segment 1c, and further, to an affinity ligand 1e, and a spacer 1d. When the substrate 1a does not have such a functional group, a functional group may preferably be introduced by an appropriate treatment. Examples of such a functional group include an amino group, hydroxyl group, carboxyl group, ester group, and chloromethyl group. However, another functional group may be used as long as it may produce the same effect.

[Block Polymer]

In the present invention, the polymer chain bound to the substrate 1a is a block polymer composed of a segment 1b having an LCST and a hydrophilic segment 1c.

First, the segment 1b having an LCST will be explained. The segment 1b having an LCST of the present invention is characteristically a polymer exhibiting an LCST in an aqueous solution. As a typical polymer constituting the segment 1b having an LCST, mention may be made of poly(N-substituted acrylamide derivatives). Other substances may be used as long as the same effect can be expected.

Then, the hydrophilic segment 1c will be explained. As the hydrophilic segment 1c, a highly hydrophilic substance may be preferably used, including polyacrylamide derivatives and polyethylene oxide (PEO). However, other substances may be used as long as the same effect can be expected.

A block polymer according to the present invention may be introduced into the substrate 1a by graft polymerization using a functional group on the surface of the substrate 1a as an initiation seed. Alternatively a free block polymer previously synthesized may be bound to the substrate 1a. In the latter case, when the block polymer contains a functional group which is capable of binding to the substrate 1a, such as an amino group, hydroxyl group, carboxyl group, and epoxy group, at the end of the segment 1b having an LCST, the functional group may be used as a group to be bound to the substrate 1a. In contrast, when the block polymer contains no functional group which is capable of binding to the substrate 1a, at the end of the temperature sensitive segment 1b, a functional group capable of binding to the substrate 1a may be newly introduced into the end of the temperature sensitive segment 1b to bind to the substrate 1a.

The block polymer to be used in the present invention can be synthesized by various polymerization methods. The most popular method is the living polymerization method. As the living polymerization method, use may be made of living anion polymerization, living cation polymerization, and living radical polymerization. These polymerization methods are preferable, since the molecular weight of a block polymer and the ratio of segment lengths can be accurately controlled to synthesize block polymers having a narrow molecular-weight distribution. However, a method of producing a block polymer according to the present invention is not limited to these. Any method may be used as long as the same effect can be expected.

[Affinity Ligand]

An affinity ligand 1e according to the present invention refers to a substance having affinity for a specific physiologically active agent. The physiologically active agent used herein refers to a substance, such as a peptide and a protein, exhibiting an activity within a living body and having an interaction with and affinity for other substances present in vivo and ex vivo. As the affinity ligand 1e according to the present invention, use may preferably made of a substance that forms a chelate ring with a target physiologically active agent via a metal ion. As examples of the substance that forms a chelate ring with a physiologically active agent, use may be made of a compound having a dicarboxylic acid structure within a molecule. However, affinity ligand 1e that does not form a chelate ring with a physiologically active agent may be used as long as the same effect can be expected.

When an affinity ligand 1e has a functional group, such as amino group, hydroxyl group, carboxyl group, and epoxy group, capable of binding to the substrate 1a or the spacer 1d within the molecule, the functional group may be used as a group binding to the substrate 1a or the spacer 1d. In contrast, when an affinity ligand 1e has no functional group capable of binding to the substrate 1a or the spacer 1d within the molecule, a functional group capable of binding to the substrate 1a or the spacer 1d may be introduced to the affinity ligand 1e to bind to the substrate 1a or the spacer 1d. In either case, care must be taken not to inactivate the affinity ligand 1e by binding to the substrate 1a and the spacer 1d.

[Spacer]

A spacer 1d according to the present invention is a chemical agent substance present between the substrate 1a and the affinity ligand 1e. The spacer 1d preferably has functional groups, such as an amino group, hydroxyl group, carboxyl group, and epoxy group, at both ends before binding to the substrate 1a and the affinity ligand 1e. A spacer 1d according to the present invention is not particularly limited as long as it can bind to the substrate 1a and the affinity ligand 1e at an appropriate distance. Specifically, it is preferable to use an ethylene glycol diglycidyl ether derivative.

[Separation/Recovery Step for Physiologically Active Agent]

Now, how to separate/recover a target physiologically active agent using a structure for separating a physiologically active agent according to the present invention will be explained.

A target physiologically active agent according to the present invention refers to a substance having affinity for the affinity ligand 1e and selectively binding to it. Generally, a peptide and a protein are used. Of them, a peptide or protein containing a histidine tag is preferable. The protein or peptide containing a histidine tag satisfactorily forms a chelate ring with the affinity ligand 1e. However, the binding between an affinity ligand 1e and a physiologically active agent is not limited to that mediated by a chelate ring. A chemical bonding (including a hydrogen bonding) may be accepted. Chemical or physical adsorption may be used as long as the same effect can be expected.

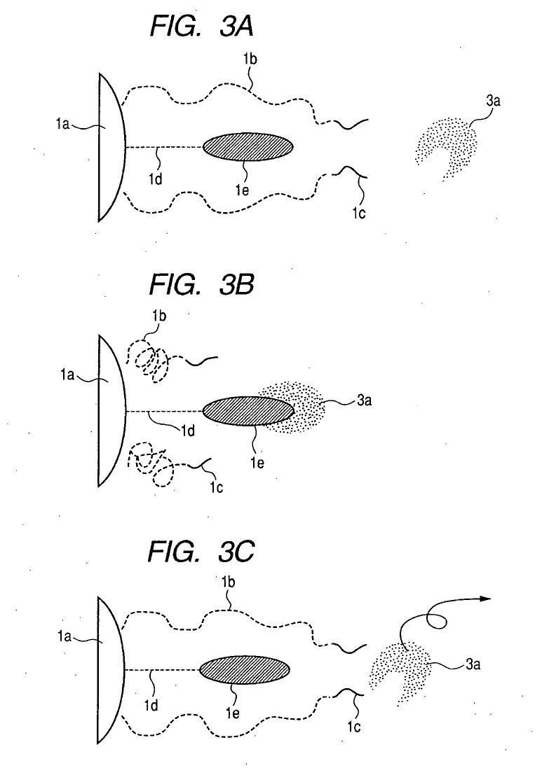

FIGS. 3A, 3B and 3C illustrated a system recovering a physiologically active agent selectively from an aqueous solution containing the physiologically active agent.

A structure for separation of a physiologically active agent illustrated in FIG. 1 is soaked in an aqueous solution containing a physiologically active agent 3a. When a temperature is equal to or higher than the LCST of the segment 1b having an LCST, the affinity ligand 1e is covered with a block polymer, which is composed of the segment 1b having an LCST and the hydrophilic segment 1c and the physiologically active agent 3a and the affinity ligand 1e thus do not bind to each other (FIG. 3A).

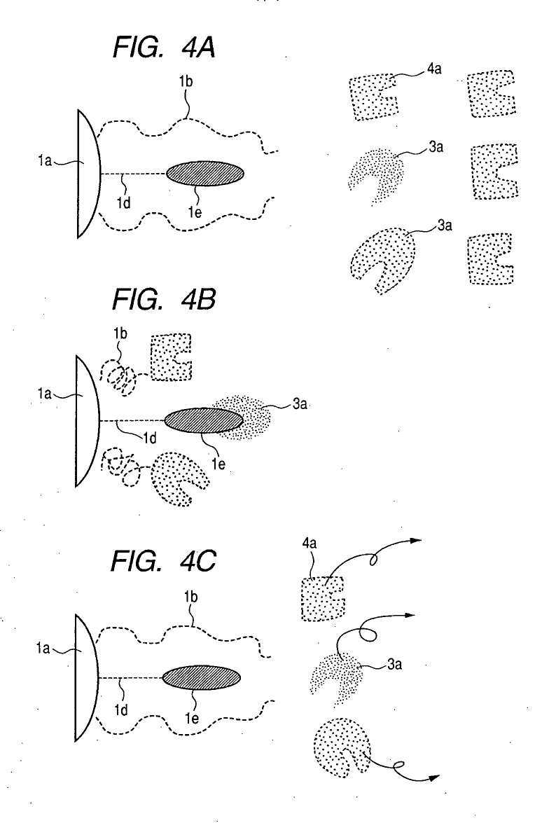

On the other hand, when the temperature is elevated to a temperature equal to or higher than the LCST, the segment 1b having an LCST becomes hydrophobic and shrinks in the solution and the affinity ligand 1e is thus exposed to bind to the physiologically active agent 3a (FIG. 3B). When a hydrophilic segment 1c is not present as is in the case shown in FIGS. 4A, 4B and 4C, the following problem occurs. The segment 1b having an LCST is hydrophobic when the temperature is equal to or higher than the LCST and adsorbs to the physiologically active agent 3a in a non-specific manner via hydrophobic interaction. On the other hand, in the present invention, since the hydrophilic segment 1c binds to the segment 1b having an LCST, the hydrophilicity of the surface of the structure for separation of a physiologically active agent does not change between before and after the temperature is elevated to the LCST. As a result, the nonspecific adsorption of the physiologically active agent 3a can be suppressed.

After impurities are washed off while the affinity ligand 1e is bound to the physiologically active agent 3a and the temperature is lowered to a temperature equal to or lower than the LCST, the segment 1b having an LCST is dissolved again in the solution. The affinity ligand 1e is covered with a block polymer composed of the segment 1b having an LCST and the hydrophilic segment 1c. Therefore, the affinity ligand 1e and the physiologically active agent 3a are dissociated due to the excluded volume effect. With this mechanism, the physiologically active agent 3a can be recovered from the solution (FIG. 3C).

The system for recovering a physiologically active agent, which is illustrated in FIGS. 3A, 3B and 3C, is advantageous, since an acidic or alkaline solution and a large amount of a competitive agent need not be added for recovering a separated physiologically active agent, unlike a conventional case, inactivation of a physiologically active agent can be suppressed to a minimum. Furthermore, since a hydrophilic segment 1c is present, nonspecific adsorption of a physiologically active agent can be suppressed. This is a great advantage of the present invention compared to the prior art.

The present invention will now be explained by way of Examples, which will not be construed as limiting the present invention.

### EXAMPLE 1

In this Example, a method for manufacturing a structure illustrated in FIG. 1 will be described and the structure is evaluated for performance of separating/recovering a physiologically active agent. In the Example, a block polymer is introduced into a substrate by immobilizing a free block polymer previously synthesized onto the surface of a substrate.

[1-1] Synthesis of a Block Polymer

A reaction solution is prepared by adding PEO with a brominated terminal (Mn: 1000, Mw/Mn: 1.1), N-isopropyl acrylamide (NIPAM), CuCl, tris[2-(dimethyl amino)ethyl]amine (Me₆TREN), and dimethylformamide (DMF) to a schlenk reaction tube. Vacuum deaeration is performed under a freeze condition to remove oxygen in the schlenk tube and atomic transfer radical polymerization (ATRP) is allowed to proceed room temperature. After a lapse of a predetermined time, a large amount of mercaptoacetic acid is added to the reaction system to carboxylate the end of a block polymer to be produced. The carboxylation is confirmed by NMR. Furthermore, the carboxylated block polymer (poly(EO-b-NIPAM)-COOH) is purified by dialysis and recovered in a dry state by lyophilization. The recovered poly(EO-b-NIPAM)-COOH is dissolved in an aqueous solution containing water-soluble carbodiimide (WSC) and reacted with N-hydroxyl succinimide (NHS) to convert the carboxyl group of the poly(EO-b-NIPAM)-COOH into an active ester group. The progress of the reaction is confirmed by NMR. The active esterified block polymer (poly(EO-b-NIPAM)-NHS) is determined by GPC. It is confirmed that the number average molecular weight is 6000 and a molecular weight distribution index is 1.3.

[1-2] Introduction of a Block Polymer into a Substrate

The active ester group of the poly(EO-b-NIPAM)-NHS is reacted with an amino group of an aminopropyl silica beads (5 μm) in water to introduce poly(EO-b-NIPAM)-NHS into the surface of the amino propyl silica beads. Introduction of the poly(EO-b-NIPAM)-NHS is confirmed by surface element analysis by XPS.

[1-3] Introduction of an Affinity Ligand into a Substrate

An epoxy group of polyethylene glycol glycidyl ether is reacted with the remainder aminopropyl silica beads having poly (EO-b-NIPAM)-NHS introduced therein in water to bind polyethylene glycol glycidyl ether. Subsequently, the amino group of iminodiacetic acid is reacted with ethylene glycol glycidyl ether in water and thereafter the reaction solution is stirred in an aqueous solution of CuCl₂ to form a chelate between iminodiacetic acid and Cu²⁺ ion. Formation of a chelate is confirmed by eluting Cu²⁺ using ethylene diamine tetra-acetic acid (EDTA) and quantifying the amount of Cu²⁺ by atomic adsorption analysis. The silica beads having both an affinity ligand and a block polymer immobilized by such a manner will be hereinafter referred to as “code 1.”

[1-4] Separation/Recovery of a Physiologically Active Agent

As a model of a physiologically active agent, bovine serum albumin (BSA) is used.

[1-4-1]

Code 1 is dispersed in distilled water set at 25° C., and further BSA is added to prepare an aqueous solution for evaluation. After the aqueous solution is shaken at 25° C. for one hour, the solution is centrifuged to precipitate code 1 and the supernatant is taken. The BSA concentration of the supernatant thus taken is evaluated by measuring absorption. It is confirmed that the initial BSA concentration does not change. From this, it is demonstrated that BSA does not adsorbed to code 1 in the aqueous solution at 25° C.

[1-4-2]

Code 1 is dispersed in distilled water set at 40° C., and further BSA is added to prepare an aqueous solution for evaluation. After the aqueous solution is shaken at 40° C. for one hour, the solution is centrifuged to precipitate code 1, and the supernatant is taken. The BSA concentration of the supernatant thus taken is evaluated by measuring absorption. It is confirmed that it is 10% or less of the initial BSA concentration. From this, it is demonstrated that BSA is selectively adsorbed to code 1 in the aqueous solution at 40° C.

[1-4-3]

After the temperature of the aqueous solution in which code 1 having BSA adsorbed thereto in step [1-4-2] is dispersed is lowered to 25° C., the solution is centrifuged to precipitate code 1 and the supernatant is taken. The BSA concentration of the supernatant thus taken is evaluated by measuring absorption. It is confirmed that the initial BSA concentration does not change. From this, it is demonstrated that BSA can be recovered by selectively adsorbing BSA to code 1 at 40° C. and then lowering the temperature to 25° C.

[1-4-4]

Comparative Example is carried out by using the silica beads (code 1′) having only a block polymer immobilized thereto, which is prepared in the steps of preparing code 1 according to Example 1 without performing Example [1-3]. Code 1′ is dispersed in distilled water set at 40° C., and further BSA is added to prepare an aqueous solution for evaluation. After the aqueous solution is shaken at 40° C. for one hour, the solution is centrifuged to precipitate code 1′ and the supernatant is taken. The BSA concentration of the supernatant thus taken is evaluated by measuring absorption. It is confirmed that the initial BSA concentration does not change. From this, it is demonstrated that BSA adsorption to code 1′ in the Example [1-4-2] is not non-specific adsorption and caused by an affinity ligand.

### EXAMPLE 2

In this Example, a method for manufacturing a structure illustrated in FIG. 1 will be described and the structure is evaluated for the performance of separating/recovering a physiologically active agent. In the Example, a block polymer is introduced into a substrate by introducing a functional group into the surface of the substrate and performing grafting polymerization using the functional group as an initiator seed.

[2-1] Pretreatment of a Substrate

Silica beads are washed with concentrated nitric acid and collected by filtration. The silica beads thus collected are heated at 135° C. for 5 hours under a dry nitrogen atmosphere and then dispersed in anhydrous toluene. To the silica beads dispersed in toluene solution, 2-(4-chloromethylphenyl)ethyl trimethoxy silane serving as a silane coupling agent is added and reacted with a hydroxyl group on the surface of the silica beads. In this manner, chloro-methylation is performed. The progress of the reaction is confirmed by surface element analysis by XPS based on C1 atom as an index. Subsequently, the silica beads thus chloro-methylated is dispersed in water. To the dispersion solution, aminoethane thiol is added to aminate a part of a chloromethyl group. Furthermore, sodium dithiocarbamate is added to the dispersion to react with the remainder chloromethyl group. The progress of the reaction is confirmed by surface element analysis by XPS based on N atom and S atom as indexes. The silica beads treated in this manner will be hereinafter referred as “pretreated beads.”

[2-2] Introduction of an Affinity Ligand into a Substrate

An epoxy group of polyethylene glycol glycidyl ether is reacted with an amino group of the surface of the pretreated beads in water to bind polyethylene glycol glycidyl ether to the surface of the pretreated beads. Subsequently, the amino group of iminodiacetic acid is reacted with ethylene glycol glycidyl ether in water. The silica beads having an affinity ligand introduced therein thus prepared will be hereinafter referred to as “affinity beads.”

[2-3] Synthesis and Introduction of a Block Polymer into a Substrate

A reaction solution is prepared by adding affinity beads, NIPAM, water, and benzyl N,N-diethyl dithiocarbamate to a schlenk reaction tube. The schlenk tube is replaced with nitrogen, and UV graft polymerization is allowed to proceed at room temperature to form poly (NIPAM) on the surface of the affinity beads. A UV lamp used herein has an irradiation wavelength within the range of 312 nm to 577 nm. Next, to the reaction system, acrylamide is added and UV graft polymerization is allowed to further proceed to form poly (NIPAM-block-AAm) on the surface of the affinity beads. The molecular weight and molecular weight distribution of a polymer chain formed from benzyl N,N-diethyldithio carbamate, which is added as a free initiation seed. It is confirmed that the number average molecular weight is about 5200 and the molecular weight distribution index is 1.25. The silica beads having both an affinity ligand and a block polymer immobilized by such a manner will be hereinafter referred to as “code 2.”

[2-4] Separation/Recovery of a Physiologically Active Agent

As a model of a physiologically active agent, lysozyme is used.

[2-4-1]

Code 2 is formed into slurry and packed into a stainless-steel column (30×4.6 mm φ). Thereafter, NiCl₂ solution is passed through to form a chelate of an iminodiacetic acid group with N²⁺ ion. Then, while maintaining a column temperature at 40° C., a predetermined amount of an aqueous lysozyme solution is passed through the column and an eluate is collected. The lysozyme concentration of the eluate thus collected is evaluated by the micro BCA method. It is confirmed that lysozyme is not contained in the eluate. From this, it is demonstrated that lysozyme can be selectively adsorbed to code 2 at 40° C.

[2-4-2]

Continued from Example 2, step [2-4-1], the column temperature is lowered to 20° C. After the aqueous lysozyme solution is passed through, the eluate is taken. The lysozyme concentration of the eluate is measured by micro BCA method. It is confirmed that the eluate contains lysozyme at a concentration not less than 90% of the initial lysozyme concentration. From this, it is demonstrated that lysozyme can be recovered by selectively adsorbing lysozyme to code 2 at 40° C. and lowering the temperature to 20° C.

[2-4-3]

Comparative Example is carried out by using the silica beads (code 2′) having only a block polymer immobilized thereto, which is prepared in steps of preparing code 2 according to Example 2 without performing step [2-2]. Code 2′ is converted into slurry and packed into a stainless-steel column (30×4.6 mm φ), and then NiCl₂ solution is passed through. Then, while the column temperature is maintained at 40° C., a predetermined amount of an aqueous lysozyme solution is passed through the column to obtain the eluate. When the lysozyme concentration of the eluate thus taken is evaluated by the micro BCA method. It is confirmed that the eluate contains lysozyme at a concentration of 100% of the initial lysozyme concentration is obtained. From this, it is demonstrated that the adsorption of lysozyme to code 2′ in the Example [2-4-1] is not non-specific adsorption but caused by an affinity ligand.

This application claims priority from Japanese Patent Application No. 2005-134985 filed on May 6, 2005, which is hereby incorporated by reference herein.

What is claimed is:

1. A structure for separation of a physiologically active agent comprising
a substrate,
a substance exhibiting affinity for the physiologically active agent, and
a block polymer composed of a segment having a lower critical solution temperature (LCST) and a hydrophilic segment,
wherein the substance exhibiting affinity and the block polymer are bound to the substrate.

2. The structure for separation according to claim 1, wherein the substance exhibiting affinity for the physiologically active agent has a structure that forms a chelate ring with the physiologically active agent.

3. The structure for separation according to claim 1, wherein the segment having an LCST is a polyacrylamide derivative.

4. The structure for separation according to claim 1, wherein the hydrophilic segment is polyethylene oxide.

5. A method for recovering a physiologically active agent comprising the steps of:
preparing the structure for separation according to claim 1 and a solution containing a physiologically active agent;
soaking the structure for separation in the solution set at a temperature equal to or lower than an LCST, thereby adsorbing the physiologically active agent to the structure for separation; and
recovering the physiologically active agent adsorbed to the structure for separation.
